(19) United States
(12) Patent Application Publication
(10) Pub. No.: US 2005/0097628 A1
(43) Pub. Date: May 5, 2005

(54) Terminological mapping

(76) Inventors: Yves Lussier, New York, NY (US); Jianrong Li, Flushing, NY (US)

Correspondence Address: BAKER & BOTTS, 30 ROCKEFELLER PLAZA, NEW YORK, NY, 10112, US

(21) Appl. No.: 10/948,423

(22) Filed: Sep. 23, 2004

Related U.S. Application Data
(63) Continuation-in-part of application No. PCT/US03/35470, filed on Nov. 6, 2003.
(60) Provisional application No. 60/424,728, filed on Nov. 6, 2002.

(51) Int. Cl. A01H 1/00

(52) U.S. Cl. 800/260

(57) ABSTRACT

The present invention relates to the systematic use of terminology and knowledge based technologies to enable high-throughput mapping between databases having different vocabularies. In particular embodiments, it may be used to map between a database having a phenotypic terminology descriptive of non-human animals and a database having a broad-coverage clinical (anthropocentric) terminology.

### SPECIFICATION

This application is a continuation-in-part of International Patent Application No. PCT/US03/35470, filed on Nov. 6, 2003, published as WO 2004/044818 on May 27, 2004, which claims priority to provisional U.S. application No. 60/424,728, filed Nov. 6, 2002, which are incorporated by reference in its entirety herein

### FIELD OF THE INVENTION

The present invention relates to the systematic use of terminology and knowledge based technologies to enable high-throughput mapping between databases using different terminologies.

### BACKGROUND OF THE INVENTION

Recent advances in molecular biology have provided increasing amounts of complex data that require novel methods of analysis. For example, the success of the human genome project has increased the need for novel bioinformatics strategies designed to map molecular functional features of gene products to complex phenotypic descriptions, such as those of genetically inherited diseases.

To date, methods for studying complex phenotypes have taken two basic approaches. The first, more traditional approach is “forward genetics,” which focuses on phenotypes and looks to find causative genes. “Knock out” animal models are the typical means for proving and analyzing traits influenced by single genes; however, more complex phenotypes affected by multiple, potentially unknown, genetic loci, as well as epistatic relations among them, require more complicated, multivariate methods of analysis. The second approach—“reverse genetics”—is a by-product of the genomic revolution, and focuses on a specific gene in order to discover its function and contextual relevance in an organism.

In addition to the advances being made in molecular biology, there is a wealth of information accumulating relating to “phenotypes,” the manifestations of genetic material. Phenotypes fall into a wide variety of uncountable categories, including molecular activities, cellular morphology, tissue structure, gross anatomical features, clinical values (e.g., blood chemistry, white blood cell count), and epidemiologic factors (e.g., risk of heart disease). In academic research, the phenotypes not infrequently are displayed in a non-human system—a bacterium, yeast, mollusk, worm, fruit fly, fish or lab mammal. The vocabularies applied refer to non-human organisms. In contrast, the vocabularies of clinical researchers apply to humans.

The respective terminologies that serve the academic and clinical medicine communities are of great importance to each individual field. However, links between the two fields are necessary, as medicine increasingly incorporates basic biological science advances into clinical practice, and biologists or bioinformaticians validate their experiments using real patient data. Comparative biological studies have led to remarkable biomedical discoveries such as evolutionarily conserved signal transduction pathways (e.g., in the worm, Caenorhabditis elegans) and homeobox genes (e.g., in the fruitfly, Drosophila melanogaster). The discoveries made by comparative biology at the molecular level illustrate the value of developing methodologies for communicating results between disparate research fields.

Recently, comparative genomic studies to elucidate conserved gene functions have made significant advances principally via complementary integrative strategies such as functional genomics and standard notations for gene or gene function (e.g., The Gene Ontology Consortium). However, there is a pressing demand of technologies for greater integration of phenotypic data and phenotype-centric discovery tools to facilitate biomedical research (Freimer and Sabatti, 2003, Nat Genet. 34(1):15-21(2003); Gerlai, 2002, Trends Neurosci. 25(10):506-9(2002); Bogue, 2003, J Appl Physiol. 94(6):2502-2509; Pool and Esnayra,. 2000, “Bioinformatics—Converging Data to Knowledge Workshop Summary. Borad on Biology”, Commission on Life Sciences. National Research Council. National Academy Press 41p; Altman and Klein, 2002, Ann Rev Pharmaco & Toxicol. 42:113-133; Botstein and Risch, 2003, Nat Genet. 33 Suppl:228-237; Collins et al., 2003, Science. 300(5617):286-290; Balmain et al., 2003, Nat Genet. 33 Suppl:238-244; Peltonen and McKusick, 2001, Science. 291(5507):1224-1229; Freimer and Sabatti, 2003, Nature Genet. 34(1):15-21). While automated technologies permit increasingly efficient genotyping of organisms' cohorts across distinct species or individuals with distinct phenotype, the ability to precisely specify an observed phenotype and compare it to related phenotypes of other organisms remains challenging (Navarro et al., 2003, Trends Biotechnol. 21(6):263-268) and does not match the throughput capabilities of genotypic studies. Further, phenotypic “qualifiers” span biological structures and functions extending from the nanometer to populations (Blois, 1984, MS. Information in Medicine: The Nature of Medical Descriptions. Berkeley, Calif.: University of California Press): proteins, organelles, cell lines, tissue, Model Organism, clinical, genetic and epidemiologic databases. This diversity of scales, disciplines and database usage (Rector et al., 2002, Proc AMIA Symp:642-646) has lead to an extensive variety of uncoordinated phenotypic notations including 1) differences in the definition of a phenotype (e.g. trait, quantitative traits, syndromes; Mahner and Kary, 1997, J Theoret Biol. 186(1):55-63), 2) differences in the terminological granularity and composition (Elkin et al., 1998, Proceedings MEDINFO, 660-664; Elkin et al., 1998, in Chute, ed., Proceedings AMIA Ann. Symp, 765-774; Mays et al., 1998, in Cimino J J, ed. Proceedings AMIA Ann Symp, 259-263; Stuart et al., 1995, MEDINFO Proc, 33-36) and 3) distinct usage of identical terms according to the context (e.g. organism, genotype, experimental design, etc.).

The heterogeneity of phenotype notation can be found in both the clinical and biological databases. While each Model Organism Database System has standardized the phenotypic notation for its own research community, bridging the gap of phenotypic data across species remains a work in progress. In this regard, the Phenotype Attribute Ontology (PAtO) is an initiative stemming from the Gene Ontology Consortium (Ashburner et al., 2000, Nat Genet 25(1):25-29) to derive a common standard for various existing phenotypic databases. In addition, the standardization of the database schema emerging from the PAtO collaboration will considerably increase the interoperability of phenotypic databases and may also clarify problems related to the terminological representation.

In contrast, while heterogeneous database systems have been shown to unify disparate representational database schema (Hucka et al., 2002, Pac Symp Biocomput. 450-461; Mork et al, 2002, Proc AMIA Symp.533-537), the semantic modeling of the notation representation remains manually edited (e.g., structural naming differences, semantic differences and content differences; Sujansky, 2001, J Biomed Inform. 34(4):285-298). In addition, these general-purpose heterogeneous database systems have not been specifically adapted to the complexity of phenotypic data reuse for comparative biology and genomics.

The most prominent barrier to the integration of heterogeneous phenotypic databases is associated with the notational (terminological) representation. While terminologies can be manually or semi-automatically integrated, as illustrated by the meta-terminologies (e.g. Unified Medical Language System), such a process is both time consuming and labor expensive (Cimino et al., 1994, JAMIA 1(1):35-50; Burgun and Bodenreider, 2001, Proc AMIA Symp 81-85). An alternative approach employing ontology (Lambrix and Edberg, 2003, Pac Symp Biocomput. 589-600; Li et al., 2000, Proc AMIA Symp 497-501), and lexicon-based mapping utilizes knowledge-based and semantic-based terminological mapping (Hill et al., 2002, Genome Res. 12(12):1982-1991; Bodenreider et al., 2001, Proc AMIA Symp. 61-65; Burgun et al., 2002, Proc AMIA Symp 86-90; Lussier et al., 2001, Proc AMIA: 418-422; Tuttle et al., 1991, Proc AMIA:219-223; Tuttle et al., 1995, MEDINFO. 8(Pt 1):162-166). While single-strategy mapping systems have demonstrated limited success (only capable of mapping 13-60% of terms;Lussier et al., 2001, Proc AMIA: 418-422; McCray et al., 1994, in Ozbolt J G, ed. Proceedings of the Eighteenth Annual Symposium in Computer Applications in Medical Care. Philadelphia: Hanley & Belfus, 235-239; Rocha et al., 1994, in Ozbolt J G, ed. Proceedings of the 18th Annual Symposium on Computer Applications in Medical Care. 690-694; Zeng and Cimino, 1996 Proc AMIA 105-109), systems using a methodical combination of multiple mapping methods and semantic approaches have demonstrated significantly improved accuracy (Cantor et al., 2003, Stud Health Technol Inform 62-67; Sarkar et al.,2003, Pac Symp Biocomput. 439-450; Cantor et al., 2003, AMIA Symposium (2003); Zeng and Cimino, 1996,. Proc AMIA Annu Fall Symp. 105-109). Zhang and Bodenreider, 2003, Proceedings of 2004 the Pacific Symposium on Biocomputing, World Scientific pp. 164-165, have explored the information extractable from anatomic ontologies not only as explicit but also as implicit semantic relationships, and have found that specific relationships can be generated by multiple techniques.

The present invention relates to an automated multi-strategy mapping method for high throughput combination and analysis of phenotypic data deriving from heterogeneous databases with high accuracy. As demonstrated by the working example provided herein, this mapping strategy also enabled the assessment of the qualitative discrepancies of phenotypic information between a clinical terminology and a phenotypic terminology.

### SUMMARY OF THE INVENTION

The present invention relates to methods of identifying related records in distinct databases, at least one of which contains terms associated with conceptual identifiers, in which (i) a term in one database is broken down into component elements; (ii) various combinations of those elements are generated; (iii) a mapping operation to the other database is performed using the element combinations; (iv) successfully mapped pairs of terms are conceptually processed to remove redundant pairs; and (v) the processed terms are then subjected to semantic processing to remove less relevant pairs. In specific, non-limiting embodiments, one of the databases includes phenotype data pertaining to non-human organisms and the other database includes human phenotype data.

The association of records according to the present invention facilitates the mining of bioinformatics data, and allows the number of relationships associated with any biodata item to be expanded as interdatabase relationships are created by terminologic mapping. Where the association of records is made via mapping of phenotype terms applied to different organisms, the new relationships identified may be added to any comparative biology already established for the organisms.

The present invention is based, at least in part, on the results of studies that demonstrated the successful mapping of terms from Phenoslim, a phenotype structured vocabulary developed by the Mouse Genome Database, and SNOMED CT, a comprehensive human clinical ontology.

In particular embodiments, the present invention may be used to map between a database having a phenotypic terminology descriptive of non-human animals and a database having a broad-coverage clinical (anthropocentric) terminology, which do not share a cross-index or a translation table. Alternatively, it can also be used to enhance the mapping between two databases that have incompletely overlapping terminologies in which some identical concepts are mapped in different terms due to the absence of a cross-index or an obsolete cross-index, and to map species taxonomies from different sources from one to the other.

### Definitions

“Biodata item” broadly refers to a piece of information pertaining to the normal or abnormal biology of a cell or organism or phenotypic data associated therewith. A biodata item may be a term, as defined below.

“Conceptual identifier” designates a characteristic of a term. As one non-limiting example, where a relational database comprises a table, and a row of the table represents a record, a column of the table is designated by a conceptual identifier. In certain non-limiting embodiments, the conceptual identifier is a metadata identifier. In other embodiments, a conceptual identifier may be separably linked to a term in a flat-file database, for example as a comma separated value. In an ontology, a conceptual identifier may be associated with several synonymous terms.

“Domain ontology” is a set of classes and associated slots that describe a particular domain (Musen, 1998, Methods of Information in Medicine 37(4-5):540-550, as cited in Oliver et al., 2002, Pacific Symposium on Biocomputing 7:65-76). It may “contain classes that are not intended to have instances, but that represent classes organized in a hierarchy to serve as a controlled vocabulary. When instances are added to classes of a domain ontology, it becomes a “knowledge base.”

“Knowledge base” is a domain ontology having classes and instances (see above).

“Ontology” is a set of related concepts “used to describe a certain reality.” (Guarino, 1998, Proceedings of FOIS '98”, Trento, Italy, Amsterdam, IOS Press, pp. 3-15, as cited in Oliver et al., 2002, Pacific Symposium on Biocomputing 7:65-76). The relationships between concepts may be simple hierarchies (in which each child has only one parent) or more complex (for example, where a child may have more than one parent). More than one ontology may be used to capture different aspects of information; for example, Gene Ontology™ uses three ontologies (molecular function, biological process and cellular structure) to organize bioinformatics data. Complex relationships may be depicted as directed acyclic graphs (DAGs). Two species of ontology are referred to herein: (1) structured vocabularies and (2) domain ontologies.

“Phenotype” is any observable characteristic of an organism, broadly construed, which is not the genotype (or part of the genotype, such as a gene or gene control element) of the organism. Accordingly, as non-limiting examples, the term “phenotype” as used herein includes protein conformation (e.g., excessive post-translational modification of an allelic variant of collagen type II at the 519 position), physico-chemical properties of a protein or other biomolecule (e.g., oxygen binding of sickle hemoglobin), the function of a cellular organelle (e.g., damaged mitochondria, as occur in certain neuromuscular diseases); cellular morphology (sickled erythrocytes), multi-cellular formations (e.g., rouleaux formation of sickled erythrocytes); tissue conformation (e.g., re-epithelialization of Barrett's esophagus); organ morphology (e.g., tetrology of Fallot); organism morphology (e.g., dwarfism); organism behavior (e.g., learning disabled, bipolar disorder); motor capabilities (e.g., ability to initiate movements, muscle tone and strength); coordination (e.g., cerebellar ataxia); sensory capabilities (e.g., anosmia); metabolic function (e.g., blood chemistries, renal function, liver function, fever); reproductive functions (e.g., sterility); dimensions (e.g.,length, width, height), weight, diagnosis of disease (e.g., Parkinson's disease, acromegaly, malaria); pathogen (e.g., human immunodeficiency virus); organism species (e.g., human, rat); geographical location (e.g., North America, Sub-Saharan Africa); population (e.g., New York City resident; Inuit); family history (e.g., family history of cardiac disease); treatment history (e.g., previous treatment with dilantin) and response to treatment (e.g., tumor refractory to vincristine). The genetic basis for the phenotype is frequently, although not always, unknown. Despite the fact that the foregoing example phenotypes largely relate to humans, phenotypes may be exhibited by any human or non-human organism, including single celled organisms, viruses, or prions.

“Record” is a linked set of biodata items. In a relational database, the record may be a row of a table. The term as used herein also encompasses linked biodata items in a non-relational (e.g. flat-file) database (e.g., comma separated values).

“Semantics” relates to the meaning, as opposed to the structure, of an expression.

“Structured vocabulary” (also “structured terminology”) means a vocabulary (terminology) that is organized according to relationships amongst its terms. For example, a structured vocabulary may be a set of terms organized according to “is a” and/or “part of” relationships. A structured vocabulary is a type of ontology.

“Term” is a character or characters that refers to a thing, method or concept. For example, a term may be a string of text. A term may comprise one or a plurality of elements. Linguistically, a term comprises at least one word. An example of a term having more than one word is “congestive heart disease,” wherein “congestive,” “heart” and “disease” are all elements of the term.

“Terminology” is used interchangeably with “vocabulary,” and is a set of terms that, in a particular context (e.g. a database), have meanings that are either expressly defined (e.g., in a glossary) or defined by usage. For example, a given database may utilize a terminology (vocabulary) where terms or phrases carry definitions which may or may not be shared by other databases. A “structured terminology” or “structured vocabulary” is a type of ontology (defined above). However, as used herein, a terminology or vocabulary is not structured unless specified.

### BRIEF DESCRIPTION OF THE DRAWINGS

FIG. 1 is a simplified block diagram of a system for generating an amalgamated database from a plurality of databases with relationships not determinable using a common index or join operation in accordance with the present invention;

FIG. 2 is a flow chart providing the method steps for a first method of generating an amalgamated database from a plurality of databases which do not have a common index or key field;

FIG. 3 is a flow chart further illustrating a method of generating an expanded term set for use in terminological mapping for identifying related concepts among multiple databases;

FIG. 4 is a flow chart further illustrating a method of performing common concept identification in accordance with the present invention;

FIG. 5 is a graph illustrating the proportion of Phenoslim concepts mapped into semantic types of SNOMED, in connection with an example of a terminological mapping process used in the present invention;

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention as defined by the appended claims.

### DETAILED DESCRIPTION OF THE INVENTION

The present invention relates to methods for mapping a first vocabulary term in a first database to a second vocabulary term in a second database, wherein at least the second database contains terms associated with conceptual identifiers, comprising the steps of (1) decomposing the first term of the first database into component elements; (2) generating a plurality of combinations of elements to produce a set of combinatorial terms; (3) performing a mapping operation to map a plurality of combinatorial terms to terms in the second database, thereby producing a set of mapped term pairs; (4) performing conceptual processing to remove any mapped term pair having the same conceptual identifier(s) as another mapped term pair to form a processed set of mapped term pairs having unique conceptual identifiers; and (5) performing semantic processing to remove any mapped term pair having an irrelevant conceptual identifier, wherein a mapped term pair of the result set allows the joining of a record associated with the first term of the first database with a record associated with the second term of the second database. In certain non-limiting embodiments, the method comprises the further step of joining the aforementioned records.

For purposes of clarity of description, and not by way of limitation, the detailed description of the invention is divided into the following subsections:
(i) databases;
(ii) preprocessing;
(iii) decomposition and generating combinations;
(iv) normalization;
(v) mapping;
(vi) conceptual processing;
(vii) semantic processing; and
(viii) uses of the invention.

### Databases

The methods of the present invention may be applied to any database, including databases that do not contain bioinformatics information but that rather pertain to other technology or art. At least one of the databases (the second or target database) used in the inventive methods contains terms that carry conceptual identifiers. In non-limiting embodiments, one or both databases are relational databases having terms that carry conceptual identifiers. In preferred embodiments, the target database contains conceptual identifiers that are organized into one or more ontology.

In preferred embodiments, the methods of invention are applied to bioinformatics databases, including databases that contain information (biodata items) relating to genes, proteins, biochemistry, cellular constituents, cellular interactions, tissues, organisms, behavior, diseases, cellular dysfunction or degeneration, etc

Specific, non-limiting examples of databases that comprise human clinical information are Quick Medical Reference™, or QMR, which is a clinical support database of diseases, signs and symptoms from First Data Bank, Inc. of Bruno, Calif., and Online Mendelian Inheritance in Man (OMIM), available from the National Center for Biotechnology Information (http://www.ncbi.nlm.nih.gov/omim/). The OMIM database provides, inter alia, genetic and genomic data and text associated with inheritable diseases. Another example is the dbSNP (for Single Nucleotide Polymorphism) database (http://www.ncbi.nlm.nih.gov/SNP/index.html). Yet another example is the mapping of databases using distinct taxonomies of species such as the Universal Virus Database of the International Committee on Taxonomy of Viruses (ICTVdB; http://www.ncbi.nlm.nih.gov/ICTVdb/) and the databases of the National Center for Biotechnology Information (“NCBI”) for GenBank (http://www.ncbi.nlm.nih.gov/Genbank/index.html). GenBank is using the NCBI taxonomy to annotate species and in the domain of viruses, the ICTVdB is considered more up-to-date than the NCBI Taxonomy, which is believed to contain misassigned taxonomies for some species:
http://www.ncbi.nlm.nih.gov/entrez/guery.fcgi?db=Taxonomy). Swissprot also contains uncoded disease terms.

Specific, non-limiting examples of databases that comprise non-human genetic and phenotypic data include:
LocusLink (http://www.ncbi.nlm.nih.gov/LocusLink/);
Mouse Genome Informatics (http://www.informatics.jax.org/);
Flybase (http://flybase.bio.indiana.edu/);
Wormbase (http://www.wormbase.org/).
the Berkely Drosophila Genome Project (http:/www.fruitfly.org/);
The Saccharomyces Genome Database (http://www.yeastgenome.org/);
The Rat Genome Database (http://rgd.mcw.edu/);
The Institute for Genomic Research (TIGR) (http://www.tigr.org/) and
The Zebrafish Information Network (http://zfin.org/cgi-bin/webdriver?MIval=aa-ZDB_home.apg), to name a few. Most of those listed in this paragraph are members of the Gene Ontology Consortium,™ which has, as a goal, the standardization of ontologies.

### Preprocessing

In specific, non-limiting embodiments of the invention, a preprocessor may be used to standardize files by taking a text or XML input and integrating semantic context with files in an XML grammar. The input may be a semantic type for each concept that may or may not have more than one associated term.

For example, but not by way of limitation, where terminologic mapping is to be used in conjunction with generation of an amalgamated database, a preprocessor may create a unique identifier for each term, a unique concept identifier, an empty slot for the preferred concept term for this concept identifier, and/or an empty slot for the semantic type (the semantic type may preferably be in the target term).

### Decomposition and Generating Combinations

According to this step, generally, a term in one database is broken down into “component elements” and then various combinations of those elements are generated. The generated combinations are referred to as a “set of combinatorial terms” or, alternatively, an “expanded term set.” Although it is not required that all combinations be generated, it is preferred.

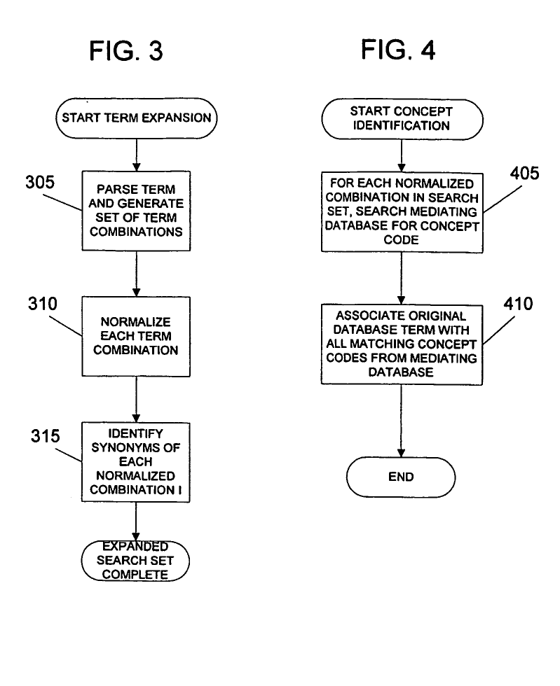

FIG. 3 is a flow chart illustrating the steps used in one exemplary algorithm for generating a set of combinatorial terms from the terms presented in the source databases. The terms identified in the source databases can include structured or non-structured text. In the case of non-structured text, a natural language preprocessing step can be applied to identify search terms for expansion. For multiple word search terms, the search term is parsed into single word components and combinations of these components are identified. For example, if the search term identified in database 1 includes a three word phrase, A-B-C, this would be parsed into the components A, B, C and combinations ABC, AB, AC, BC, A, B, and C would be established.

In a specific, non-limiting embodiment of the invention, two subsystems may be applied: (1) concatenation breakdown and (2) decomposition into terminologic components. Concatenation breakdown analyses the phrase and if it finds a regular division pattern across all terminological entries (e.g. class: subclass, class>sub-sub-class>sub-sub class or term1, term2, term3, term4 . . . ) of n divisions, it will unchain the concatenation and create n+1 rows: the original full term and the n separate rows for each subset (components). For decomposition in terminological components, each component is comprised of one string of one or more word and, for those strings that have more than one word, every combination of words is generated and each combination occupies a new row.

### Normalization

The identified combinational terms are preferably subjected to a normalization operation (step 310), although this step is not required and the method may be applied to non-normalized terms. In preferred, non-limiting embodiments of the invention, the target terms in the second database may also be normalized, and preferably both combinatorial term and target term are normalized. Normalization is a process by which the terms are transformed into a common format. For example, terms can be placed in an order depending on the part of speech ( i.e., verb, noun, adjective, etc.), capitalization can be removed, plural forms replaced with non-plural forms and the like. Known lexical tools such as NORM, which is a component available in UMLS, can be used to normalize the terms for the expanded term set. As its name implies, Norm converts text strings into a normalized form, removing punctuation, capitalization, stop words, and genitive markers. Following the normalization process, the remaining words are sorted in alphabetical order. For example, “Hemophilia B” from OMIM becomes “b hemophilia.”

### Mapping

Mapping may be performed by any method known in the art. Conventional mapping methods include exact match of the terms or term components, and partial mappings or relaxation methods allowing, for example, for typographical errors or international spelling differences (e.g. “hemoglobin” vs. haemoglobin”) in the term components. For example, Krauthammer has described a system “using approximate text string matching techniques (Krauthammer et al., 2000, Gene 259(1-2):245-252). His “system is a dictionary-based system that recognizes spelling variations in names, while keeping the reference to the closest nearest match.”. The product of the mapping set is a set of mapped pairs of term components from a “set of combinatorial terms,” where each pair contains a combinatorial term from the first database and a term from the second database.

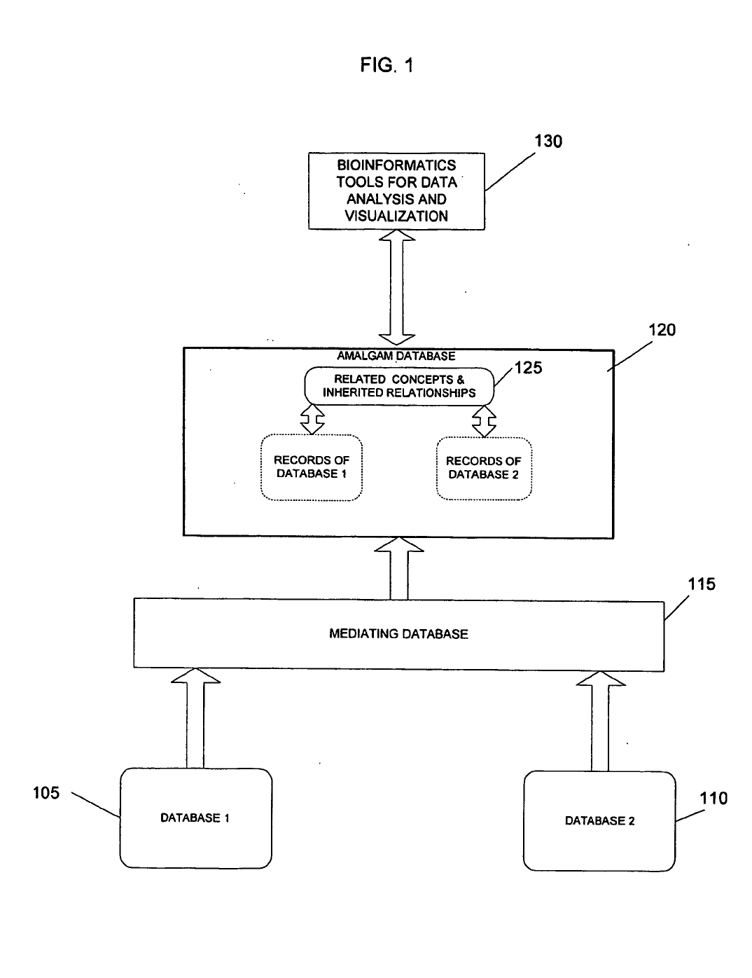

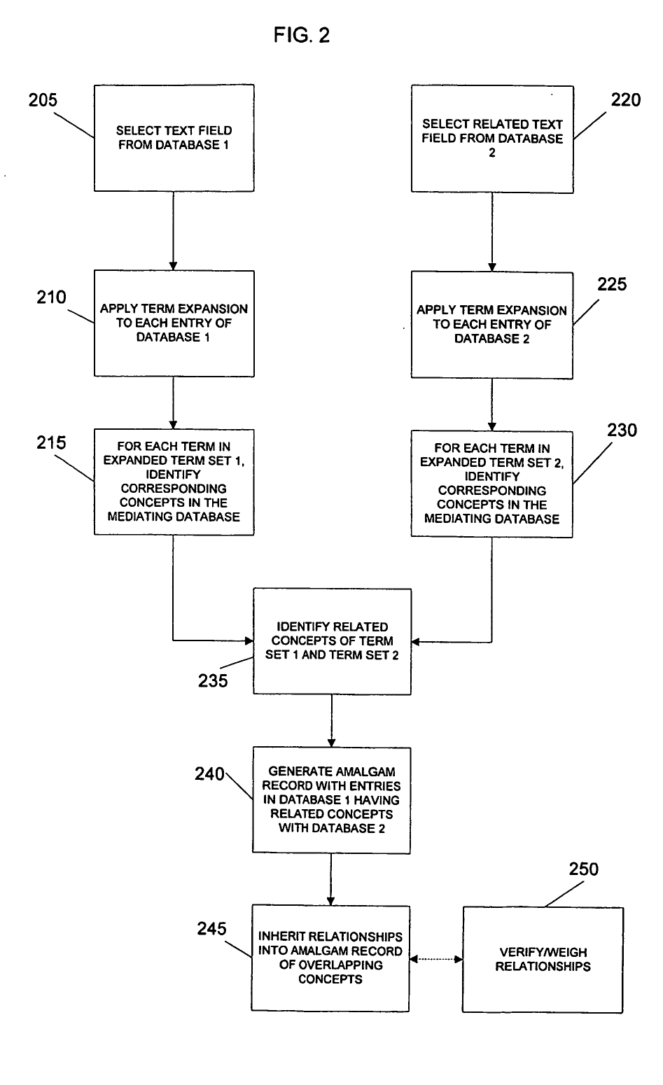

In non-limiting embodiments of the invention, mapping may be performed by creating an amalgamated database, as set forth in International Patent Application No. PCT/US03/35470, published as WO 2004/044818, and as schematically depicted in FIGS. 1 and 2 and as described below.

Briefly, FIG. 1 is a simplified block diagram illustrating the generation of an amalgam database from records of two or more databases using relationships that go beyond the use of a common index or common key. Referring to FIG. 1, two source databases are shown, database 1 105 and database 2 110. It is assumed that database 1 105 and database 2 110 contain information which is somewhat related but do not share a common key or index field which would enable a direct JOIN operation to be performed to allow interoperability between the records of the two databases.

Database 1 105 and database 2 110 are coupled to a mediating database 115. Mediating database 115 can be a single database or a plurality of interoperable databases. The meditating database 115 is used to identify related concepts between database 1 105 and database 2 110 such that data in these two distinct databases can be rendered interoperable in the resulting amalgam database 120. The mediating database 115 generally provides an overarching ontology from which concepts can be identified from at least one datafield in each of database 1 and database 2.

Preferably, terminological mapping is applied to at least one of database 1 or database 2 and the mediating database 115 to identify related concepts. In addition to an overarching ontology from which related concepts can be identified, the mediating database 115 can also provide relationships associated with the related concepts.

The relationships of the related concepts in the mediating database 115 can be inherited into the amalgam database 120 such that a new family of relationships can emerge between the records of database 1 and those of database 2 110. This is illustrated in sub-box 125 which pictorially illustrates the newly identified set of related concepts and inherited relationships establishing an interoperable link between at least a set of records in database 1 105 and database 2 110. From the set of related concepts and inherited relationships, additional inferential relationships, not expressly stated in any of database 1 105, database 2 110 or the mediating database 115, can also be established within the amalgam database 120. Thus, the mediating database 115 is capable of operating more than as a mere cross index or foreign key between the first database 1 105 and database 2 110.

Relationships among the records of database 1 and database 2 can be explored by recursive mapping. For example all ancestors of a concept identified from database 1 105 can be found in the mediating database 115 by navigation the relevant “parent-child” relationships. In a like manner, parent-child relationships of the concept can also be identified in database 2 110. Through an evaluation of these ancestral relationships, a set of overlapping relationships it may be uncovered. Thus, a concept of database 1 105 may be associated with an ancestry relationship with a record of database 2, even though the mediating database may not contain a direct relationship linking the concepts of database 1 to database 2 with only one “parent-child” relationship.

FIG. 2 is a flow chart illustrating a process for generating an amalgam database 120 in accordance with the present invention. In step 205 a user selects a text field from database 1 105 which contains text-based information of interest. For example, database 1 may include a TERM column, in which semi-structured or unstructured text is used to describe the database entries. In the context of the present invention, semi-structured text is that which follows a set of rules with respect to vocabulary, order and syntax. Unstructured text does not require compliance with any normalization criteria. An example of unstructured text wold include abstracts of articles.

In step 215, the terms in the expanded term set from step 210 are used to identify a first set of concepts in the mediating database 115. As further illustrated in FIG. 4, concepts can be identified in the mediating database by finding matches to the terms in the expanded term set with those in the mediating database and associating a concept identifier in the mediating database with the matching terms. Steps 210 and 215 can be viewed as terminological mapping which will return a “match” for similar terms which do not necessarily present an exact match to the term in the original database.

In the most generalized case, database 2 110 (FIG. 1) does not contain direct references to the concept code identifiers of the mediating database and cannot be directly joined to the mediating database 115 through traditional database 115 operations. In this case, steps 220, 225 and 230 are performed in order to map terms of database 2 110 to the concepts of the mediating database 115. Steps 220, 225 and 230 are similar to those described above with respect to steps 205, 210 and 215, respectively. In those cases where database 2 110 includes an association with the concepts of the mediating database 115, the process of FIG. 2 can advance to step 235.

Following steps 215 and 230, at least a subset of the terms of database 1 105 and database 2 110 have been mapped to a set of one or more concept identifiers of the mediating database 115 (FIG. 4, step 405). From these individual mappings, those records of database 1 having a related concept identifier with records of database 2 are identified and those records are associated by the mediating database concept identifier in step 235 (FIG. 4, step 410). A table can be generated in the amalgam database in step 240 which is indexed or keyed by the concept identifier from the mediating database 115. From the set of related concepts identified in step 240, the relationships in the mediating database associated with those concepts can also be inherited into a table in the amalgam database 120 (step 245).

Optionally, additional processing can be applied to verify or assign weights to the term-concept relationships that are derived in the amalgam database (step 250). For example, term-concept relationship tuples can be searched in a database of articles related to the subject matter, such as Medline, to determine if there is substantial co-occurrence of the term-concept pair in published works. Term-concept pairs which do not have a sufficient co-occurrence ranking can be dropped or given a lower weighting. Further, established information retrieval weighing techniques may be used to stratify results such as term frequency * inverse document frequency (TF*IDF) (Hersh, 2003, A Health and Biomedical Perspective, Series: Health Informatics, 2nd Edition, XIV, ISBN: 0-387-95522-4, Springer). It will be appreciated that co-occurrence analysis is but one method that can be used to evaluate the strength of the concepts and relationships in the amalgam database 120.

The order of preference for mapping, in nonlimiting embodiments of the invention, is as follows (from most to relatively least preferred): (1) a full term match which is an exact match without decomposition; (2) normM matches without decomposition; (3) exact matches between a component of a decomposed term of the first databse and a term of the second; (4) norm matches between a component of a decomposed term of the first database and a term of the second database; (5) imprecise approximate match (allowing for typographical errors) of a component of a full term of the first databse and a term of the second database; and (6) imprecise approximate match (allowing for typographical errors) of a component of a full term of the first database and a term of the second database.

### Conceptual Processing

Once a set of mapped pairs has been created, members of the set may be conceptually processed to remove redundant pairs, to form a “processed set of mapped term pairs.”

Where combinatorial terms are generated based on a term of the first database, if the term of the first database carries a conceptual identifier, all the generated combinatorial terms carry the same conceptual identifier. Accordingly, the steps of conceptual and semantic processing are applied to the conceptual identifiers of the term from the second database in any mapped pair.

Where only the second of the two databases contains terms having conceptual identifiers, a conceptual identifier associated with a given mapped term pair may then be compared to the conceptual identifier of another mapped term pair, and if both mapped term pairs have the same conceptual identifier, one term pair is discarded. This comparison may be performed among a plurality, and preferably all, members of the set of mapped pairs.

Where both databases contain terms associated with conceptual identifiers, in one embodiment of the invention, both conceptual identifiers (e.g., P,Q, where the first value (here, P) is the conceptual identifier of the term from the first database and the second value (here,Q) is the conceptual identifier of the term from the second database) of a given mapped pair are compared to the conceptual identifiers of another mapped pair, and if both conceptual identifiers between pairs match (e.g., P,Q=P′,Q′, where prime (′) denotes identifiers from the second pair) one pair is discarded. Of note, the conceptual identifier of the first term is always the same. Alternatively, the system can be designed to compare only the conceptual identifiers of the terms from the second database, and reject pairs having redundant concept identifiers. Such comparisons may be made between a plurality of members of the set of mapped pairs, and preferably between all pairs.

### Semantic Processing

A plurality of members of the processed set of mapped pairs may then be subjected to semantic processing, which comprises one or both of the sub-processes: (i) semantic inclusion criteria, and (ii) subsumption, preferably in that order. This step (or series of sub-steps) is designed to increase the relevancy of the information retrieved.

Semantic inclusion criteria are a set of rules or conditions regarding what concepts should be included in the final set of mapped term pairs. For example, but not by way of limitation, a set of concepts that are desirably and/or necessarily present in all mapped term pairs may be predetermined. Conversely, and also considered “inclusion criteria” herein, certain concepts that are not to be present may also be identified. By specifying semantic inclusion criteria, the present invention avoids the retention of less relevant mapped term pairs in the result set. Such irrelevant pairs may arise, in one non-limiting instance, through homonymy; for example, in collecting data regarding malignant melanoma, one wants to include a transformed nevus but exclude the mole that burrows in the garden. The set of concepts permitted may not include, or may exclude, “non-human animal” or “endogenous host” or “animal.”

The set of inclusion criteria may be made more or less stringent, depending on the objectives of the operator.

The determination of the inclusion criteria may performed manually, knowing the concepts present in one or both databases, and the association between concepts and concept identifiers may either be performed manually or may be determined using a mediating database or metathesaurus (e.g., the UMLS Metathesaurus (http://www.nlm.nih.gov/pubs/factsheets/umlsmeta.html). The concept identifiers for included or excluded information may be used to select or reject mapped term pairs of the processed set, based on the concept identifier associated with the term of the second database.

The subprocess of subsumption requires that the conceptual identifier(s) associated with the term(s) of each mapped pair be organized into an ontology, which can be a structured vocabulary or domain ontology/knowledge base. In certain instances, for example, where the second database is part of the Gene Ontology Consortium, or is itself a structured vocabulary (e.g., Phenoslim) the conceptual identifiers are already organized into ontologies. In others, it may be necessary to manually or by the operation of a computer organize concept identifiers of the mapped pairs according to an ontology. This organization may be performed using the set of mapped pairs or may be performed on concept identifiers of the second database prior to mapping.

In non-limiting embodiments, an ancestor-descendant table reflecting hierarchical relationships (e.g., “is-a” or “is part of”) may be constructed. Focusing on the concept identifiers of the terms from the second database in a plurality of mapped pairs, ancestors that subsume other descendant concepts are removed, based on the hypothesis that most specific match is also the most relevant.

The product of the semantic processing step is the result set. The result set contains mappings between the original term of the first database and one or more terms of the second (target) database. Each map is assigned a classification outcome: exact conceptual match between the original full term and a target term of the target database or “classification” under the term in the target database.

In preferred non-limiting embodiments of the invention, the semantic step may comprise assessing, for semantic validity, each mapping pair between a term or a component of a term decomposition of the first database with a term of the second database, identified by the following methods, in decreasing order of preference: (1) a full term match which is an exact match without decomposition; (2) nornM matches without decomposition; (3) exact matches between a component of a decomposed term of the first databse and a term of the second; (4) norm matches between a component of a decomposed term of the first database and a term of the second database; (5) imprecise approximate match (allowing for typographical errors) of a component of a full term of the first databse and a term of the second database; and (6) imprecise approximate match (allowing for typographical errors) of a component of a full term of the first database and a term of the second database. For pairs identified at different levels (1-6), moving down the preference list, if a semantically valid pair is identified at a particular level (e.g. 2), additional pairs identified at lower levels (e.g., 3-6) may be disregarded (as the increasing levels progressively relax the stringency of the mapping and therefore are more likely to be erroneous maps).

### Uses of the Invention

In preferred specific non-limiting embodiments of the invention, the present invention may be used to map one structured vocabulary to another, as illustrated by the working example set forth below. By mapping terms—for example terms describing categories—in the two structured vocabularies, information, such as biodata items, associated with the terms may be linked. In particularly preferred embodiments, phenotype categories reflected by two distinct structured vocabularies may be mapped. Once phenotype categories from two distinct databases are mapped, the records associated with the phenotype categories of both databases may be joined.

### EXAMPLE

### Terminological Mapping

An automated multi-strategy mapping method for high throughput combination and analysis of phenotypic data deriving from heterogeneous databases with high accuracy has been developed. The method includes a mapping strategy that provides for the assessment of the qualitative discrepancies of phenotypic information between an anthropocentric clinical terminology and a non-human animal phenotypic terminology.

The method made use of Phenoslim, SNOMED and UMLS. Phenoslim is a particular subset of the phenotype vocabularies developed by Mouse Genome Database (MGD) that is used by the allele and phenotype interface of MGD as a phenotypic query mechanism over the indexed genetic, genomic and biological data of the mouse. The 2003 version of PS containing 100 distinct concepts was used in the current study.

SNOMED CT terminology (version 2003) is a comprehensive clinical ontology that contains about 344,549 distinct concepts and 913,697 descriptions, which are test string variants for a concept. SNOMED-CT satisfies the criteria of controlled computable terminologies and, in addition, provides an extensive semantic network between concepts, supporting polyhiearchy and partonomy as directed acyclic graphs (DAGs) and twenty additional types of relationships. It also contains a formal description of “roles” (valid semantic relationships in the network) for certain semantic classes. SNOMED CT has been licensed by the National Library of Medicine for perpetual public use as of 2004 and will likely be integrated to UMLS.

UMLS is created and maintained by the National Library of Medicine. The 2003-version of the UMLS consisting of about 800,000 unique concepts and relationships taken from over 60 diverse terminologies was used in this example. In addition, UMLS includes a curated semantic network of about 120 semantic types overlying the terminological network. Moreover, at the time of this example, UMLS contained an older version of SNOMED (SNOMED 3.5, 1998) that houses about half the number of concepts and descriptions of the current version of SNOMED-CT. The relationships found in the source terminologies in UMLS are not curated. Thus transformations over the unconstrained UMLS network are required to obtain a DAG and to control convoluted terminological cycles.

Norm is a lexical tool available from the UMLS. As its name implies, Norm converts text strings into a normalized form, removing punctuation, capitalization, stop words, and genitive markers. Following the normalization process, the remaining words are sorted in alphabetical order.

The applications and scripts pertaining to implementation of the methods for this example were written in Perl and SQL, although other computer languages could be used without limitation. The database software used was IBM DB2 for workgroup, version 7. The Norm component of the UMLS Lexical Tools was obtained from the National Library of Medicine in 2003. Applications were run on a Dual-processor SUN UltraSparc III V880 under the SunOS 5.8 operating system.

Phenoslim was mapped to SNOMED CT to develop an architecture that integrates lexical, terminological/conceptual and semantic approaches to methodically take advantage of pre-coordination and post-coordination mechanisms. The specific method steps used sequentially were a) decomposition of Phenoslim concepts in components, b) normalization of Phenoslim and SNOMED CT, c) mapping of PS components to SNOMED CT, d) conceptual processing, and e) semantic processing. Steps a), b) and c) are “term processing” steps that have been separated for clarity. Retired concepts and descriptions of SNOMED were not used in the study, though they are present in the SNOMED files. The method steps a-e used in this example are described more fully below.

Step a—Decomposition of Phenoslim concepts in components. Each Phenoslim concept is represented by one unique text string consisting of several words. Every combination of word was generated for each unique text string (including the full string) and mapped back to the original concept. A terminological component (TC) is a string of text consisting of one of these combinations.

Step b—Normalization of Phenoslim and SNOMED CT. Each terminological component of Phenoslim and each term associated with a SNOMED CT concept (SNOMED descriptions) was normalized using Norm (ref. material section).

Step c—Mapping of PS components to SNOMED CT. Each normalized TC was mapped against each normalized SNOMED description using the DB2 database.

Step d—Conceptual Processing. This process simplifies the output of the mapping methods. The Conceptual Processor is a database method that identifies all distinct pairs of conceptual identifiers of Phenoslim and SNOMED CT (PS-CT Pairs) that have been mapped by the previous terminological processes.

Step e—Semantic Processing. The semantic processing consists of two successive subprocesses: (i) semantic inclusion criteria, and (ii) subsumption. For inclusion criteria, mapped SNOMED CT concepts were sorted according to the criteria “that they must be a descendant of at least one semantic class” as shown in Table 1. This process eliminates erroneous pairs arising from homonymy of terms due to the presence of a variety of semantic classes in SNOMED that are irrelevant to phenotypes. An inclusion criteria was chosen since valid concepts may inherit multiple semantic classes. The list of SNOMED codes related PS concept was further reduced by subsumption with the relationships found in the relationship table of SNOMED as follows: two ancestor-descendant tables (one from the “is-a” relationship of the relationship table of SNOMED CT and another one from the partonomy relationships “is part of”) were constructed. Each network of SNOMED CT concepts paired to a unique PS concept was then recursively simplified by removing “is-a” ancestors that subsume other concepts of the network concept, based on the hypothesis that most specific match is also the most relevant. The same procedure was repeated for the “is part of” relationship. Further, additional relationships of the disease and finding categories were explored in the relationship table and the concept related to a disease or finding was considered subsumed and then removed (within the scope of SNOMED concepts paired to the same PS concept). The remaining set of PS-CT pairs were considered valid for the evaluation.

TABLE 1
Included Semantic Classes of SNOMED CT

| | SNOMED CT Concept |
|---|---|
| Concept Identifier | Name |
| 257728006 | Anatomical Concepts |
| 118956008 | Morphologic Abnormality |
| 64572001 | Disease (disorder) |
| 363788007 | Clinical |
| | history/examination |
| 246188002 | Finding |
| 246464006 | Functions |
| 105590001 | Substance |
| 243796009 | Context-dependent |
| | categories |
| 246061005 | Attribute |
| 254291000 | Staging and scales |
| 71388002 | Procedure |
| 362981000 | Qualifier value |

The mapping methods previously described produce from zero to multiple putative SNOMED concepts every Phenoslim concept. Every group of distinct SNOMED concepts related to a unique PS concept was further assessed according to the following criteria: (i) classification—the SNOMED CT concepts are valid classifier or descriptor of part of the Phenoslim concept (Good/Poor), (ii) identity—the meaning of the SNOMED CT concept is exactly the same as that of the Phenoslim concept, (iii) completeness of representation of the meaning by SNOMED concepts, (iv) redundancy of representation of SNOMED concepts, (v) presence of erroneous matches. In addition, SNOMED CT was searched to find an identical identifier or a class that could represent every PS concept that was not paired using the automated method. The efficacy of the mapping method using precision and recall was measured.

Using the term expansion and mapping methods described herein, every combination of words contained in each term associated with the 100 concepts of Phenoslim were computed yielding 4,016 terminological components. These components were processed in Norm by every possible mapping with a SNOMED-CT description calculated in DB2 in less than 2 minutes (about 3,5 billion possible pairs). 4,842 distinct terminological pairs were found. The conceptual processing reduced this number to 1,387 pairs between Phenoslim and SNOMED CT concepts. The final semantic processing provided the final set consisting of 740 distinct pairs (426 pairs did not meet the semantic inclusion criteria and 221 pairs were removed by subsumption).

Three Phenoslim concepts were not mapped, one of which could not be mapped or classified in SNOMED CT (the only true negative map). Referring to Table 2 below, seventy-nine (79) PS concepts were fully mapped to a valid composition of SNOMED concepts, fifteen (15) of which also contained one erroneous and superfluous SNOMED code. Eighteen (18) PS concepts were incompletely mapped, two of which also contained an erroneous and superfluous concept. Overall, eighteen (18) concepts were also redundantly mapped (not shown in the table)—having more than one representation of the same concept or an overlapping group of concepts.

TABLE 2
Evaluation of the Quality of the Mapping between each
Group of SNOMED Concepts associated to each Concept of Phenoslim

Validity of the
Mapping to a
Cluster of
SNOMED
Concepts

| | | Valid | False |
|---|---|---|---|
| Phenoslim's | Complete Map | 64 | 15 |
| Concepts | (identity and | | |
| Mapped by | classification) | | |
| the present | Incomplete Map | 18 | 2 |
| methods | (classification) | | |

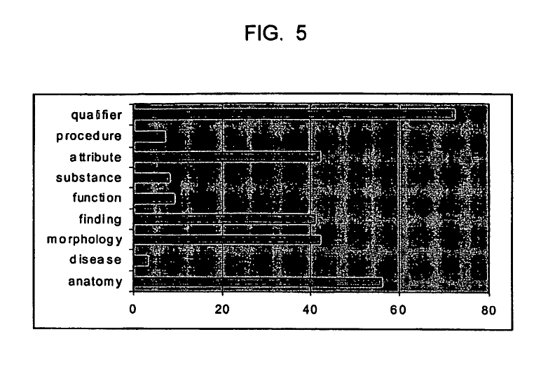

FIG. 5 shows the proportion of Phenoslim concepts that can be classified to the semantic types of SNOMED. On average each concept is mapped to 2.9 semantic classes.

Norm and the conceptual processing performed together at a precision of 11% (TP=64+18, FP=15+426+221). The precision of terminological classification accuracy of the methods described herein is 98% (TP=725, FP=15). The precision and recall of the present methods to classify Phenoslim concepts in SNOMED CT are 85% and 98%, respectively (TP=64+18, FP=15, FN=2); while the accuracy scores are 67% (precision) and 97% (recall) for the present methods used to map the full meaning in SNOMED (TP=64, FP=15+18, FN=2).

TABLE 3
Examples of Problematic Mappings

| Mapping | Examples | |
|---|---|---|
| Problem | Phenoslim | SNOMED |
| (i) erroneous | “ . . . premature | “immature” + “death” |
| mapping | death” | |
| (ii) partial | “Hematology . . . ” | Partially mapped |
| mapping | | missing |
| | | “hematological |
| | | system” |
| (iii) relevant | “ . . . postnatal | “postneonatal death” |
| mappings omitted | lethality”” | |
| by M³ | | |
| (iv) redundancy | “coat: hair texture | “hair texture (body |
| | defects” | structure)”, “Texture |
| | | of hair (observable |
| | | entity), Hair texture, |
| | | function (observable |
| | | entity) |
| (v) ambiguity | “renal system . . . ”, | Including the |
| | | bladder, the |
| | | urogenital? |
| (vi) inconsistency | “neurological/behavioral: . . . movement | |
| | anomalies” | |
| | “neurological/behavioral: . . . nociception | |
| | abnormalities” | |
| (vii) Not in | “Coat . . . ”, | — |
| SNOMED | “Vibrissae . . . ” | |
| (viii) Context/ | “Embryonic . . . ” | “Fetal . . . ” + |
| Representation | | “Embryonic . . . ” |
| Scope | | |

Table 3 illustrates examples of mapping problems encountered. Erroneous mapping occurred due in part to slightly different meanings of related concepts which were taken out of their context. For example, the concepts “human fetus” (>8 wks gestation) and “human embryo” (<8 wks) are subsumed by the concept “mammalian embryo” (vertebrate at any stage of development prior to birth). In SNOMED, the parent of the terms fetus and embryo is “developmental body structure” which is the one desired for mapping this mammalian concept. In addition, SNOMED is used for human and veterinary purposes, thus the representation of “embryo” may require reengineering as well. The absence of “unaccompanied” adjectival forms of anatomical locations and systems likely contributed to a large number of the partial mapping problems.

In contrast to SNOMED CT, SNOMED 98 in the current UMLS version contains adjectives mapped to the anatomical structure for corneal, skeletal, cellular, etc. In SNOMED CT, these adjectival forms are “accompanied” of the qualifier “structure” or “system structure” or “entire” as in “skeletal system”, “skeletal system structure” or “entire skeleton”. With additional semantic information in the phenotype terminology (e.g., anatomical location, or system), one could easily pre-process and extend terms with this contextual information before submitting them to Norm. Some redundancy can be solved by enriching SNOMED CT with a complete network of relationship: “the entire central nervous system” does not have a partonomy relationship with the “entire nervous system” which led to an overlap of mapping. More specifically for phenotypes of model organisms and genetics, the following concepts are incompletely conceptualized in SNOMED: “normal embryogenesis”, “tumor resistance”, “tumor sensitivity”, or “maternal effect”.

It is expected that a careful modeling of semantic criteria could further improve the accuracy of the present methods but may require machine learning approaches to avoid overtraining. For example, to further discriminate between completely and incompletely mapped concepts, a phenotype should have an anatomical local coded or explicitly mapped from the relationships of its coded concept. Context and scale from the source terminology can be processed as additional semantic criteria: phenotypes from the yeast should map to cellular and smaller SNOMED concepts, etc.

Various publications are cited herein, the contents of which are hereby incorporated by reference in their entireties.

1. A method for mapping a first vocabulary term, having a plurality of elements, in a first database to a second vocabulary term in a second database, wherein at least the second database contains terms associated with conceptual identifiers, comprising the steps of (1) decomposing the first term of the first database into component elements; (2) generating a plurality of combinations of elements to produce a set of combinatorial terms; (3) performing a mapping operation to map a plurality of combinatorial terms to terms in the second database, thereby producing a set of mapped term pairs; (4) performing conceptual processing to form a processed set of mapped term pairs having unique conceptual identifiers; and (5) performing semantic processing to remove any mapped term pair having an irrelevant conceptual identifer, wherein a mapped term pair of the result set allows the joining of a record associated with the first term of the first database with a record associated with the second term of the second database.

2. The method of claim 1, wherein one database is a relational database.

3. The method of claim 1, wherein both databases are relational databases.

4. The method of claim 1, wherein the second database contains conceptual identifiers that are organized into at least one ontology

5. The method of claim 1, 2, 3 or 4, wherein the term of the first database and the term of the second database refer to phenotype.

6. The method of claim 5, wherein the term of one database refers to a phenotype of a non-human animal and the term of the other database refers to a human phenotype.

7. The method of claim 1 comprising, as an additional step performed prior to step (1), preprocessing to standardize files.

8. The method of claim 1, wherein step (1) comprises the sub-step of concatenation breakdown.

9. The method of claim 1, comprising the additional step of normalizing a combinatorial term prior to mapping.

10. The method of claim 1 or 9, comprising the additional step of normalizing a term of the second database prior to mapping.

11. The method of claim 1, wherein semantic processing step (5) comprises retaining a mapped pair if it meets conditions set as semantic inclusion criteria.

12. The method of claim 1, wherein semantic processing step (5) comprises the subprocess of subsumption.

13. The method of claim 1, wherein, prior to applying the subprocess of subsumption, conceptual identifiers of mapped term pairs are organized according to an ontology.

14. The method of claim 4, wherein semantic process step (5) comprises the subprocess of subsumption.
